(19) United States
(12) Patent Application Publication
(10) Pub. No.: US 2005/0203326 A1
(43) Pub. Date: Sep. 15, 2005

(54) Process for containment of catalyst particles in a oxygenate-to-olefin process

(76) Inventors: Lawrence W. Miller, Palatine, IL (US); John J. Senetar, Naperville, IL (US)

Correspondence Address: JOHN G TOLOMEI, PATENT DEPARTMENT;UOP LLC, 25 EAST ALGONQUIN ROAD, P O BOX 5017, DES PLAINES, IL, 60017-5017, US

(21) Appl. No.: 11/014,043

(22) Filed: Dec. 16, 2004

(51) Int. Cl. C07C 1/20

(52) U.S. Cl. 585/640; 585/800; 585/921

(57) ABSTRACT

The present invention provides a process and apparatus for removing catalyst fines from a reactor effluent stream withdrawn from an oxygenate conversion reactor. This process comprises exposing a hydrocarbon stream to a catalyst within an oxygenate conversion reactor to produce a reactor effluent stream, wherein said reactor effluent stream contains catalyst fines, passing the reactor effluent stream to a filtration means, wherein said filtration means removes said catalyst fines from said reactor effluent stream, then sending said catalyst fines to a collector and then sending said catalyst fines from said collector to said oxygenate conversion reactor or discarding said catalyst fines.

### FIELD OF THE INVENTION

The present invention relates generally to a method of catalyst conservation in an Oxygenate-To-Olefin (OTO) Process utilizing a fluidized oxygenate conversion zone and a relatively expensive catalyst containing an ELAPO molecular sieve wherein catalyst losses in the product effluent stream withdrawn from the fluidized oxygenate conversion zone are significantly reduced by the use of a barrier filter to remove catalyst particles from the reactor effluent.

### BACKGROUND OF THE INVENTION

A major portion of the worldwide petrochemical industry is concerned with the production of light olefin materials and their subsequent use in the production of numerous important chemical products via polymerization, oligomerization, alkylation and the like well-known chemical reactions. Light olefins include ethylene, propylene and mixtures thereof. These light olefins are essential building blocks for the modern petrochemical and chemical industries. The major source for these materials in present day refining is the steam cracking of petroleum feeds. For various reasons, including geographical, economic, political and diminished supply considerations, the art has long sought a source other than petroleum for the massive quantities of raw materials that are needed to supply the demand for these light olefin materials. A great deal of the prior art's attention has been focused on the possibility of using hydrocarbon oxygenates and more specifically methanol as a prime source of the necessary alternative feedstock. Oxygenates are particularly attractive because they can be produced from such widely available materials as coal, natural gas, recycled plastics, various carbon waste streams from industry and various products and by-products from the agricultural industry. The art of making methanol and other oxygenates from these types of raw materials is well established and typically involves the use of one or more of the following procedures: (1) manufacture of synthesis gas by any of the known techniques typically using a nickel or cobalt catalyst followed by the well-known methanol synthesis step using relatively high pressure with a copper-based catalyst; (2) selective fermentation of various organic agricultural products and by-products in order to produce oxygenates; or (3) various combinations of these techniques.

Given the established and well-known technologies for producing oxygenates from alternative non-petroleum raw materials, the art has focused on different procedures for catalytically converting oxygenates such as methanol into the desired light olefin products. These light olefin products that are produced from non-petroleum based raw materials must of course be available in quantities and purities such that they are interchangeable in downstream processing with the materials that are presently produced using petroleum sources. Although many oxygenates have been discussed in the prior art, the principal focus of the two major routes to produce these desired light olefins has been on methanol conversion technology primarily because of the availability of commercially proven methanol synthesis technology. A review of the prior art has revealed essentially two major techniques that are discussed for conversion of methanol to light olefins. The first of these MTO processes is based on early German and American work with a catalytic conversion zone containing a zeolitic type of catalyst system. Representative of the early German work is U.S. Pat. No. 4,387,263 which was filed in May of 1982 in the U.S. without a claim for German priority. This '263 patent reports on a series of experiments with methanol conversion techniques using a ZSM-5-type of catalyst system wherein the problem of DME recycle is a major focus of the technology disclosed. Although good yields of ethylene and propylene were reported in this '263 patent, they unfortunately were accompanied by substantial formation of higher aliphatic and aromatic hydrocarbons which the patentees speculated might be useful as an engine fuel and specifically as a gasoline-type of material. In order to limit the amount of this heavier material that is produced, the patentees of the '263 patent proposed to limit conversion to less than 80% of the methanol charged to the MTO conversion step. This operation at lower conversion levels necessitated a critical assessment of means for recovering and recycling not only unreacted methanol but also substantial amounts of a DME intermediate product. The focus then of the '263 patent invention was therefore on a DME and methanol scrubbing step utilizing a water solvent in order to efficiently and effectively recapture the light olefin value of the unreacted methanol and of the intermediate reactant DME.

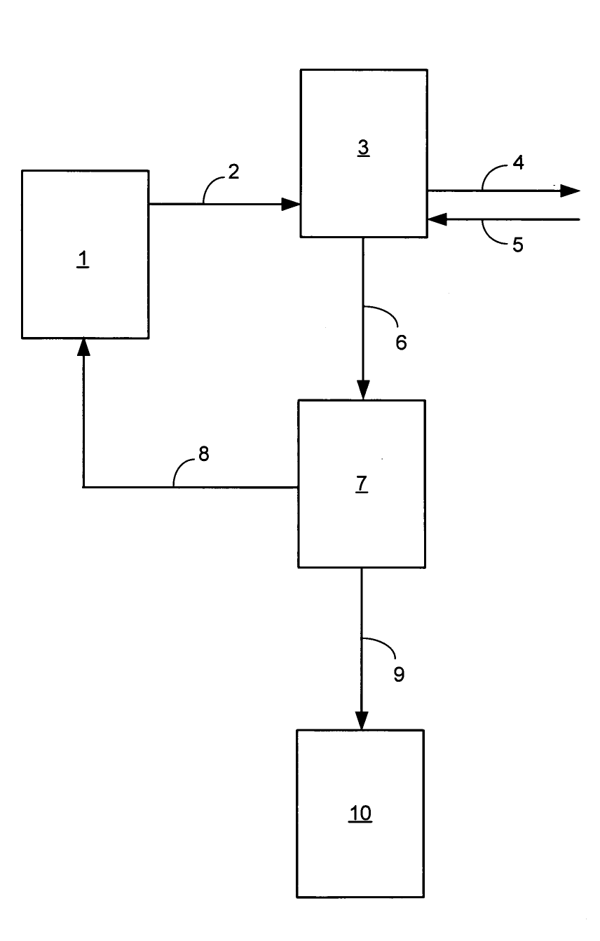

This early MTO work with a zeolitic catalyst system was then followed up by the Mobil Oil Company who also investigated the use of a zeolitic catalyst system like ZSM-5 for purposes of making light olefins. U.S. Pat. No. 4,587,373 is representative of Mobil's early work and it acknowledged and distinguished the German contribution to this zeolitic catalyst based MTO route to light olefins. The inventor of the '373 patent made two significant contributions to this zeolitic MTO route the first of which involved recognition that a commercial plant would have to operate at pressure substantially above the preferred range that the German workers in this field had suggested in order to make the commercial equipment of reasonable size when commercial mass flow rates are desired. The '373 patent recognized that as you move to higher pressure for the zeolitic MTO route in order to control the size of the equipment needed for commercial plant there is a substantial additional loss of DME that was not considered in the German work. This additional loss is caused by dissolution of substantial quantities of DME in the heavy hydrocarbon oil by-product recovered from the liquid hydrocarbon stream withdrawn from the primary separator. The other significant contribution of the '373 patent is manifest from inspection of the flow scheme presented in FIG. 2 which prominently features a portion of the methanol feed being diverted to the DME absorption zone in order to take advantage of the fact that there exist a high affinity between methanol and DME thereby downsizing the size of the scrubbing zone required relative to the scrubbing zone utilizing plain water that was suggested by the earlier German work.

Primarily because of an inability of this zeolitic MTO route to control the amounts of undesired C₄⁺ hydrocarbon products produced by the ZSM-5 type of catalyst system, the art soon developed a second MTO conversion technology based on the use of a non-zeolitic molecular sieve catalytic material. This branch of the MTO art is perhaps best illustrated by reference to UOP's extensive work in this area as reported in numerous patents of which U.S. Pat. No. 5,095,163; U.S. Pat. No. 5,126,308 and U.S. Pat. No. 5,191,141 are representative. This second approach to MTO conversion technology was primarily based on using a catalyst system comprising a non-zeolitic molecular sieve, generally a metal aluminophosphate (ELAPO) and more specifically a silicoaluminophosphate molecular sieve (SAPO), with a strong preference for a SAPO species that is known as SAPO-34. This SAPO-34 material was found to have a very high selectivity for light olefins with a methanol feedstock and consequently very low selectivity for the undesired corresponding light paraffins and the heavier materials. This ELAPO catalyzed MTO approach is known to have at least the following advantages relative to the zeolitic catalyst route to light olefins: (1) greater yields of light olefins at equal quantities of methanol converted; (2) capability of direct recovery of polymer grade ethylene and propylene with considerably less processing required to separate ethylene and propylene from their corresponding paraffin analogs; (3) sharply limited production of by-products such as stabilized gasoline; (4) flexibility to adjust the product ethylene-to-propylene weight ratios over the range of 1.5:1 to 0.75:1 by minimal adjustment of the MTO conversion conditions; and (5) significantly less coke make in the MTO conversion zone relative to that experienced with the zeolitic catalyst system.

For various reasons well articulated in UOP's patents, U.S. Pat. No. 6,403,854; U.S. Pat. No. 6,166,282 and U.S. Pat. No. 5,744,680 (all of the teaching of which are hereby specifically incorporated by reference) the consensus of the practitioners in this OTO or MTO art points to the use of a fluidized reaction zone along with an associated fluidized regeneration zone as the preferred commercial solution to the problem of effectively and efficiently using an ELAPO or SAPO-type of catalyst system in this type of service. As is well-understood by those of skill in the fluidization art, the use of this technology gives rise to a substantial problem of solid-vapor separation in order to efficiently separates the particles of the fluidized catalyst from the vapor products of the OTO or MTO reaction as well as from any unreacted oxygenate materials exiting the OTO or MTO conversion zone. Standard industry practice for accomplishing this difficult separation step involves its use of one or more vapor-solid cyclonic separating means which are well illustrated in the sole drawing of U.S. Pat. No. 6,166,282 where a series of three cyclonic separation means are used to separate spent OTO or MTO catalyst from the product effluent stream. As is clear from the teachings of these three UOP patents as well as the teachings of U.S. Pat. No. 6,121,504 and U.S. 2003/0088136 these still remain a very substantial problem of OTO or MTO catalyst contamination of the product effluent stream withdrawn from the fluidized conversion zone.

Despite the promising developments associated with the ELAPO or SAPO catalyzed routes to light olefins there are still substantial hurdles to overcome before an economically attractive OTO or MTO process can be fully realized. One very substantial economic problem is associated with the amount of fresh catalyst that must be added to the OTO or fluidized conversion zone in order to maintain the catalyst inventory in the OTO conversion system at design levels when the product effluent stream from the OTO conversion zone contains substantial amounts of contaminating catalyst particles which in the processes of the prior art discussed above are not recovered and recycled to the OTO conversion zone. This problem of effluent contamination by catalyst particles is made more significant in the non-zeolitic catalyzed route to the desired light olefins because of the relatively expensive nature of the ELAPO or SAPO molecular sieves used therein compared to the corresponding zeolitic molecular sieve, ZSM-5, which has been used and exemplified in many of the prior art OTO conversion processes. Current economic conditions are such that the cost of an equivalent amount of an ELAPO-containing catalyst system is expected to differ from the cost of the prior art zeolitic system by a factor of about 5 to 40 even considering the expected substantial savings in costs that will be associated with the large scale production of ELAPO molecular sieve for this particular application. The problem addressed by the present invention is then to provide a method for recovery and recycle of these effluent-contaminating catalyst particles that are present in the product effluent stream withdrawn from an OTO conversion zone that utilizes a fluidized transport bed system in combination with a relatively expensive ELAPO molecular sieve-containing catalyst system. In other words, the problem addressed by the present invention is to staunch the loss of catalyst particles from a fluidized OTO conversion zone operated with a relatively expensive catalyst system containing an ELAPO molecular sieve in order to decrease the consumption of the relatively expensive catalyst system and thereby improve the economics of the resulting OTO or MTO conversion process.

The present invention is carried out in a fluidized bed reactor. The effluent from the reactor will contain some catalyst fines, despite efforts to efficiently design the reactor cyclone system. These fines present a disposal problem. They will appear in the first condensed phase of the reactor effluent and have a significant negative effect upon the product quality. In addition, these catalyst fines can cause operational and maintenance problems through plugging of the instrumentation and erosion of equipment. In addition, it is undesirable to lose a significant amount of catalyst in the reactor effluent due to the value of the catalysts employed in this process. One way to remove the fines from the process would be through filtration of the initial condensate. However, there is water content in this phase. Caustic is injected into this phase to neutralize the small amount of acetic acid byproduct. The caustic addition would permanently deactivate the catalyst. Therefore, the recovered fines would be suitable only for landfill.

The solution envisioned and provided by the present invention to this catalyst loss problem involves the use of a barrier filter to remove catalyst particles from the reactor effluent.

### SUMMARY OF THE INVENTION

The present invention provides a process for converting an oxygenate to light olefins. The improved process comprises using a barrier filter to remove catalyst fines from the reactor effluent. These catalyst fines can then be returned to the reactor or sent to a spent catalyst hopper, as appropriate.

In one embodiment, the instant invention is a process for the catalytic conversion of a feedstream containing an oxygenate to light olefins which uses a fluidized conversion zone and a relatively expensive fluidized catalyst containing an ELAPO molecular sieve with recovery and recycle of contaminating catalyst particles from the product effluent stream withdrawn from the fluidized conversion zone. In the first step of the process the feedstream is contacted with the fluidized catalyst in the fluidized conversion zone at conversion conditions effective to form a mixture of partially deactivated catalyst particles and olefinic reaction products. In the second step, at least a portion of the partially deactivated catalyst particles is separated from the resulting mixture in a vapor-solid separating zone containing one or more vapor-solid cyclonic separating means operated at separating conditions effective to form a stream of partially deactivated catalyst particles and a conversion zone product effluent stream containing light olefins, unreacted oxygenates, H₂O, other reaction products and undesired amounts of contaminating catalyst particles. In the third step, the resulting product effluent stream is passed to a filtering zone and therein a stream of catalyst particles are removed from the product effluent stream. In the fourth step at least a portion of the stream of partially deactivated catalyst particles separated in the second step is passed to a regeneration zone and therein contacted with an oxidizing gas stream under oxidizing conditions effective to form a stream of regenerated catalyst particles. In the last step then the stream of freshly regenerated catalyst particles recovered from the regeneration step is recycled to the OTO conversion zone.

A highly preferred embodiment of the present invention comprises an OTO conversion process as described above in the first embodiment wherein the oxygenate present in the feedstream is methanol or dimethylether or a mixture thereof and wherein the ELAPO molecular sieve is a SAPO molecular sieve having its crystal structure corresponding to SAPO-34 or SAPO-17.

The present invention provides a process and apparatus for removing catalyst fines from a reactor effluent stream withdrawn from an oxygenate conversion reactor. This process comprises exposing a hydrocarbon stream to a catalyst within an oxygenate conversion reactor to produce a reactor effluent stream, wherein said reactor effluent stream contains catalyst fines, passing the reactor effluent stream to a filtration means, wherein said filtration means removes said catalyst fines from said reactor effluent stream, then sending said catalyst fines to a collector and then sending said catalyst fines from said collector to said oxygenate conversion reactor or discarding said catalyst fines.

Another embodiment of the present invention comprises a catalyst conservation system comprising an oxygenate conversion reactor having at least one outlet for passage of a reactor effluent. There is provided a reactor effluent filtration means to remove catalyst particles from said reactor effluent wherein said outlet is in fluid communication with said reactor, a subsystem to return a portion of said catalyst particles to the oxygenate conversion reactor either directly or to a regeneration zone to contact the catalyst with an oxidizing gas stream under oxidizing conditions sufficient to form a stream of regenerated catalyst particles and a second subsystem to remove a second portion of said catalyst particles for purposes of disposal.

### BRIEF DESCRIPTION OF THE DRAWING

The FIGURE displays the reactor effluent filter within the context of the relevant portion of a methanol to olefins plant.

### DETAILED DESCRIPTION OF THE INVENTION

This invention comprises a process for the catalytic conversion of a feedstock comprising one or more aliphatic hetero compounds comprising alcohols, halides, mercaptans, sulfides, amines, ethers, and carbonyl compounds or mixtures thereof to a hydrocarbon product containing light olefinic products, i.e., C₂, C₃ and/or C₄ olefins. The feedstock is contacted with a silicoaluminophosphate molecular sieve at effective process conditions to produce light olefins. Silicoaluminophosphate molecular sieves which produce light olefins are generally employable in the instant process. The preferred silicoaluminophosphates are those described in U.S. Pat. No. 4,440,871.

The term “light olefins” as used herein means ethylene, propylene and mixtures thereof. The expression “ELAPO” molecular sieve means a material having a three-dimensional microporous framework structure of AlO₂, PO₂ and ELO₂ tetrahedral units having the empirical formula:
(ELₓAl_{y}P_{z})O₂
where EL is a metal selected from the group consisting of silicon, magnesium, zinc, iron, cobalt, nickel, manganese, chromium and mixtures thereof, x is the mole fraction of EL and is at least 0.005, y is the mole fraction of Al and is at least 0.01 z is the mole fraction of P and is at least 0.01 and x+y+z=1. When EL is a mixture of metals, x represents the total amount of the metal mixture present. Preferred metals (EL) are silicon, magnesium and cobalt with silicon being especially preferred. The expression “SAPO molecular sieve” means an ELAPO molecular sieve wherein the EL element is silicon as described in U.S. Pat. No. 4,440,871. The expression “OTO” process means a process for converting an oxygenate to light olefins and in a preferred embodiment when the oxygenate is methanol the OTO process is referred to as an MTO process herein. The term “oxygenate” means an oxygen-substituted aliphatic hydrocarbon preferably containing 1 to 4 carbon atoms. In the instant process the feedstream comprises an oxygenate. As used herein, the term “oxygenate” is employed to include alcohols, ethers, and carbonyl compounds (aldehydes, ketones, carboxylic acids, and the like). The oxygenate feedstock preferably contains from 1 to about 10 carbon atoms and, more preferably, contains from 1 to about 4 carbon atoms. Suitable reactants include lower straight or branched chain alkanols, and their unsaturated counterparts.

In accordance with the process of the present invention, an oxygenate feedstock is catalytically converted to hydrocarbons containing aliphatic moieties such as—but not limited to—methane, ethane, ethylene, propane, propylene, butylene, and limited amounts of other higher aliphatics by contacting the aliphatic hetero compound feedstock with a preselected catalyst.

The oxygenate conversion process of the present invention is preferably conducted in the vapor phase such that the oxygenate feedstock is contacted in a vapor phase in a reaction zone with a molecular sieve catalyst at effective conversion conditions to produce olefinic hydrocarbons, i.e., an effective temperature, pressure, WHSV and, optionally, an effective amount of diluent, correlated to produce olefinic hydrocarbons. The process is affected for a period of time sufficient to produce the desired light olefin products. The oxygenate conversion process is effectively carried out over a wide range of pressures, including autogenous pressures. At pressures between about 0.001 atmospheres (0.76 torr) and about 1000 atmospheres (760,000 torr), the formation of light olefin products will be affected although the optimum amount of product will not necessarily form at all pressures. The preferred pressure is between about 0.01 atmospheres (7.6 torr) and about 100 atmospheres (76,000 torr). More preferably, the pressure will range from about 1 to about 10 atmospheres. The temperature which may be employed in the oxygenate conversion process may vary over a wide range depending, at least in part, on the selected molecular sieve catalyst. In general, the process can be conducted at an effective temperature between about 200° and about 700° C.

In the oxygenate conversion process of the present invention, it is preferred that the catalysts have relatively small pores. Preferably, the small pore catalysts have a substantially uniform pore structure, e.g., substantially uniformly sized and shaped pore with an effective diameter of less than about 5 angstroms. Suitable catalyst may comprise non-zeolitic molecular sieves and a matrix material.

The catalysts which can be used in the instant invention are any of those described in U.S. Pat. Nos. 4,440,871; 5,126,308 and 5,191,141 which are hereby incorporated by reference. Especially preferred SAPOs include the SAPO-34 and SAPO-17.

The preferred oxygenate conversion catalyst may be, and preferably is, incorporated into solid particles in which the catalyst is present in an amount effective to promote the desired hydrocarbon conversion. In one aspect, the solid particles comprise a catalytically effective amount of the catalyst and at least one matrix material, preferably selected from the group consisting of binder materials, filler materials, and mixtures thereof to provide a desired property or properties, e.g., desired catalyst dilution, mechanical strength, and the like to the solid particles. Such matrix materials are often, to some extent, porous in nature and may or may not be effective to promote the desired hydrocarbon conversion. Filler and binder materials include, for example, synthetic and naturally occurring substances such as metal oxides, clays, silicas, aluminas, silica-aluminas, silica-magnesias, silica-zirconias, silica-thorias, silica-berylias, silica-titanias, silica-alumina-thorias, silica-alumina-zirconias, alumino-phosphates, mixtures of these and the like. If matrix materials, e.g., binder and/or filler materials, are included in the catalyst composition, the non-zeolitic molecular sieves preferably comprise about 1 to 99 wt-%, more preferably about 5 to about 90 wt-% and still more preferably about 10 to about 80 wt-% of the total composition. The preparation of solid particles comprising catalyst and matrix materials is conventional and well known in the art and, therefore, need not be discussed in detail herein.

During the oxygenate conversion reaction, a carbonaceous material, i.e., coke, is deposited on the catalyst. During the conversion process a portion of the coked catalyst is withdrawn from the reaction zone and regenerated to remove at least a portion of the carbonaceous material and returned to the oxygenate conversion reaction zone. Depending upon the particular catalyst and conversion, it can be desirable to substantially remove the carbonaceous material e.g., to less than 1 wt-%, or only partially regenerate the catalyst, e.g., to from about 2 to 30 wt-% carbon. Preferably, the regenerated catalyst will contain about 0 to 20 wt-% and more preferably from about 0 to 10 wt-% carbon. Additionally, during regeneration there can be oxidation of sulfur, and in some instances nitrogen compounds along with the removal of metal materials from the catalyst. Moreover, regeneration conditions can be varied depending upon catalyst used and the type of contaminant material present upon the catalyst prior to its regeneration. The details concerning the conditions for regeneration are known to those skilled in the art and need not be further disclosed herein.

The oxygenate conversion process of the instant invention will be further illustrated in terms of a methanol-to-olefin (MTO) process which produces light olefins including ethylene and propylene from methanol. The reaction products which are withdrawn from the MTO reactor must be cooled and separated from water, a byproduct of the conversion, in a quench tower before the olefin products are recovered. In the quench tower, most of the water is condensed and the light hydrocarbons and light oxygenates are removed from the top of the quench tower as an overhead stream and the water is removed from the bottom of the quench tower. Water removed from the quench tower comprises some dissolved light hydrocarbons and heavy byproducts including heavy oxygenates including alcohols and ketones which have a normal boiling point greater than or equal to water and which must be removed by stripping the water heavy byproducts with light gases such as steam or nitrogen. In a preferred embodiment of the present invention, the reactor effluent is first sent to a quench tower to remove a water slipstream and then the remaining reactor effluent goes to a product separator where the bulk of the product water is condensed and removed. Such a two stage system is described in U.S. Pat. No. 6,403,854, incorporated by reference herein in its entirety. The feedstream passed to an MTO reactor can be refined methanol (essentially pure), or raw methanol containing water comprising up to about 30 wt-% water. The feedstream is heated and vaporized prior to being charged to the fluidized bed MTO reactor. This requires a considerable amount of energy. Therefore, it is necessary to recover as much as energy of the reactor effluent and use it to heat and vaporize the feedstream. However, water is substantially the only condensation product in the quench tower. Thus, the operating temperatures within the quench tower closely approach the bubble/dew point of pure water at the operating pressure. Although methanol and water have a boiling point differential of over 16° C. (60° F.), there is a difference in operating pressure between the methanol vaporization and the water condensation stages. This differential is due to the pressure drop through heat exchangers, the MTO reactor, piping, etc. This pressure differential results in closing the difference between the feed vaporization and product condensation temperatures, making meaningful heat exchange difficult. The presence of any water in the methanol feed, depresses the boiling point curve and exacerbates the problem. Because it is difficult to completely vaporize the feedstream using only indirect heat exchange between the feedstream and the reactor effluent, a considerable amount of external heat provided by heating the feedstream with steam is required to insure that the feedstream is fully vaporized prior to introducing the feedstream to the reaction zone. The reaction zone can comprise either a fixed bed or a fluidized reaction zone, but a fluidized reaction zone is preferred.

### DETAILED DESCRIPTION OF THE INVENTION

The present invention provides a process and system for the collection or conservation of catalyst fines from a reactor. In particular, the invention is useful in the design of a methanol to olefins plant that includes a fluidized bed type reactor. The effluent from such reactors will contain some catalyst fines, regardless of the efficiency of the design of the reactor cyclone system. In these reactors, the reactor effluent is initially in a vapor phase until condensed. While it would be possible to filter the catalyst fines from the initial condensate, a neutralizing agent such as a caustic, ammonia or an amine is introduced during this stage to neutralize any acidic byproduct. Caustic are less expensive than the amines, but they have the undesirable effect of deactivating the catalyst and making it unsuitable for further use.

The presence of fines in the reactor effluent presents a disposal problem. In addition, their presence in the first condensed phase following exit from the reactor provides a negative effect upon product quality. Catalyst fines also present operational and maintenance problems through the erosion of equipment and the detrimental effect upon instrumentation. The MTO catalyst is more expensive that the catalyst used in many other processes and accordingly, it is desirable to reuse that catalyst within the reactor. The use of a barrier filter in the present invention has been found to be particularly advantageous.

In other designs, it has been known to use a cyclone to separate out particles such as catalyst fines. However, through age and erosion of surfaces by these particles, performance of cyclones will degrade. Erosion can lead to holes forming in the cyclone with loss of catalyst. A barrier filter has been found herein to be an effective, relatively inexpensive way to capture catalyst fines. The barrier filter can be selected to handle an increased load of catalyst fines. The pressure required surface area of the filter is dependent upon the pressure drop that the filter is exposed to during its operation. Another factor to be considered in the design in the filter is the frequency of the cleaning cycle. An increased gas flow is periodically introduced to remove particles that have accumulated against the filter. Such particles, comprising catalyst fines can be divided into a portion to be returned to the reactor and a second portion to be discarded from the reactor. The gas that is used to remove particles is selected from the group consisting of nitrogen, steam and light hydrocarbons.

An occasional, but serious, problem that occurs in fluid catalytic processes is the loss of large amounts of catalyst from a vessel, such as a reactor, usually as a result of a significant mechanical failure or a radical change in operating conditions. The catalyst losses from such an event can range from minor, up to a loss of the entire contents of the vessel. A barrier filter will at least contain the catalyst during the occurrence of such an event. This would prevent the catalyst from passing into the wastewater where it would be lost from further reuse.

There are a variety of ways that a reactor effluent filter can be introduced to filter a reactor effluent stream. In one embodiment of the present invention, the filter may be located in a cyclone positioned to receive the reactor effluent. The filter may comprise a variety of materials, but the preferred filters are sintered metal filters. Sintered metal mesh filters and sintered metal powder filters may be employed in the present invention. Such filters may be purchased from Pall Corporation, East Hills, N.Y., USA.

### DETAILED DESCRIPTION OF THE DRAWING

In the FIGURE is shown the reactor filtration means 3 within a section of an oxygenate to olefins plant. A reactor 1 is shown wherein an oxygenate feedstock is contacted to a catalyst and converted to a mixture comprising light olefins and other hydrocarbons as well as some of the catalyst. This mixture is the reactor effluent that exits reactor 1 through line 2 and passes to a vessel 3 that contains at least one filter that traps catalyst particles from the reactor effluent. The reactor effluent then passes through line 4 to other parts of the oxygenate to olefins plant (not shown) for further processing including separation of the desired propylene and ethylene products from the reactor effluent. A line 5 is shown through which a gas selected from the group consisting of nitrogen, steam and light hydrocarbons is introduced into said vessel to disperse catalyst fines and any other particles from the reactor effluent filter within the vessel. This reactor effluent filter has a pore size selected to be of an appropriate size to trap essentially all of the catalyst fines. The gas introduced through line 5 sends the catalyst fines through line 6 optionally to a classifier 7 such as a cyclone. In some embodiments of the invention the catalyst fines are recycled to the reactor to avoid excessive processing of the catalyst fines. In other embodiments of the invention, the classifier is designed to separate the catalyst fines into two portions. The first portion is sent through line 8 back to reactor 1. This first portion comprises catalyst fines that are of a size that is still useful in functioning as a catalyst within reactor 1. A second portion of the catalyst fines including catalyst that is no longer useful as catalyst is sent through line 9 to waste container 10 or otherwise exits the plant. It is anticipated that additional components may be employed for the purpose of removing the catalyst fines from the reactor effluent and recycling or disposing of the removed catalyst fines. Such additional components may include additional filters, pumps, collection means and transportation means for transporting the catalyst fines within a catalyst conservation system.

1. A process of removing catalyst fines from a reactor effluent stream withdrawn from an oxygenate conversion reactor, said process comprising:
a) exposing a hydrocarbon stream to a catalyst within an oxygenate conversion reactor to produce a reactor effluent stream, wherein said reactor effluent stream contains catalyst fines;
b) passing the reactor effluent stream to a filtration means, wherein said filtration means removes said catalyst fines from said reactor effluent stream;
c) then sending said catalyst fines to a collector; and
d) then sending said catalyst fines from said collector to said oxygenate conversion reactor or discarding said catalyst fines.

2. The process of claim 1 wherein said filtration means comprises at least one vessel containing at least one filter wherein a flow of gas passes through said filter and a majority of said catalyst fines contained in said reactor effluent stream collect on said filter.

3. The process of claim 2 wherein said filter comprises sintered metal or ceramic.

4. The process of claim 2 wherein said filter has a pore size designed to remove essentially all of said catalyst fines from said reactor effluent stream.

5. The process of claim 1 wherein said catalyst fines are sent from said collector to a classification means, wherein said classification means divides said catalyst fines into a first portion having a size above a predetermined value to be sent to returned to said oxygenate conversion reactor and a second portion having a size below said predetermined value to be sent to a discarded catalyst storage container.

6. The process of claim 3 wherein said sintered metal filter is made from sintered metal powder.

7. The process of claim 2 wherein an increased gas flow is periodically introduced into said filtration means to disperse collected catalyst fines from said filter and to move said catalyst fines to said collector.

8. The process of claim 7 wherein said increased gas flow is aimed directly toward the catalyst fines.

9. The process of claim 7 wherein said gas flow directs said catalyst fines so that at least a portion of said catalyst fines return to said reactor.

10. The process of claim 7 wherein said gas is selected from the group consisting of nitrogen, steam and light hydrocarbons.

11. A catalyst conservation system comprising:
a) an oxygenate conversion reactor having at least one outlet for passage of a reactor effluent;
b) a reactor effluent filtration means to remove catalyst particles from said reactor effluent wherein said outlet is in fluid communication with said reactor;
c) a subsystem to return a portion of said catalyst particles to said oxygenate conversion reactor; and
d) a subsystem to remove a second portion of said catalyst particles for purposes of disposal.

12. The catalyst conservation system of claim 11 wherein said reactor effluent filtration means comprises at least one sintered metal or ceramic filter.

13. The catalyst conservation system of claim 11 wherein said filtration means has a pore size designed to remove essentially all of said catalyst particles from said reactor effluent.

14. The catalyst conservation system of claim 11 wherein periodically a flow of air is introduced into said filtration means to disperse said catalyst particles from said filtration means and to move said catalyst particles to a classification means.

15. The catalyst conservation system of claim 14 wherein said classification means divides said catalyst fines into a first portion having a size above a predetermined value to be sent to returned to said oxygenate conversion reactor and a second portion having a size below said predetermined value to be sent to a discarded catalyst storage container.
